(19) United States
(12) Patent Application Publication
(10) Pub. No.: US 2026/0096928 A1
(43) Pub. Date: Apr. 9, 2026

(54) DIRECT VIEW SCLERAL CONTACT LENS

(71) Applicant: Ocular Instruments, Inc., Bellevue, WA (US)

(72) Inventors: Mark A. Latina, Bellevue, WA (US); Raymond D. Graham, Bellevue, WA (US)

(73) Assignee: Ocular Instruments, Inc., Bellevue, WA (US)

(21) Appl. No.: 19/349,249

(22) Filed: Oct. 3, 2025

Related U.S. Application Data
(60) Provisional application No. 63/703,760, filed on Oct. 4, 2024.

(51) Int. Cl. A61F 9/009; A61F 9/008

(52) U.S. Cl. CPC A61F 9/009; A61F 9/008; A61F 2009/00891

(57) ABSTRACT

A direct view sclera contact lens suitable for use in direct selective laser trabeculoplasty (DSLT) procedures. With use of the direct view sclera contact lens, DSLT can be carried out with conventional SLT lasers that are commonly available. The direct view sclera contact lens comprises a lens body having an inferior end that defines a light exit surface. The light exit surface extends inwardly toward the optical axis and in a superior direction to form a compression annulus. The compression annulus contacts an eye of the patient during direct selective laser trabeculoplasty. The lens body includes a ring of evenly spaced alignment markers disposed in the light exit surface.

### CROSS-REFERENCE TO RELATED APPLICATION

This application claims the benefit of U.S. Provisional Application No. 63/703,760, filed Oct. 4, 2024, the disclosure of which is hereby incorporated by reference in its entirety.

### TECHNICAL FIELD

Embodiments of the present disclosure generally relate to ophthalmic treatment of human eyes and more specifically to the treatment of glaucoma using a laser beam. Certain embodiments generally relate to a direct view sclera contact lens for use in direct selective laser trabeculoplasty (DSLT) in which the laser beam is directed at the trabecular meshwork of the human eye through a contact lens.

### BACKGROUND

Glaucoma is a disease in which vision is impaired as a result of damage to the optic nerve or retina, and is responsible for about 25% of blindness in developed countries. A common contributor to this damage is elevated pressure of the fluid, known as the aqueous humour, within the eye. The increased intra-ocular pressure causes progressive death of retinal ganglion cells and damages axons that transfer visual information to a brain via an optic nerve.

The aqueous fluid is constantly and slowly replaced by the body with the ingress coming from a ciliary body just beneath the iris and a balancing drainage taking place through an annular spongy tissue, known as a trabecular meshwork around the edge of the iris where it meets the cornea which transitions into the sclera. Drainage takes place from the meshwork through to a structure called Schlemm's canal and eventually into the body's circulatory system.

The primary cause of elevated pressure in an eye is due to an imbalance between the ingress and egress of fluid due to malfunctioning of the annular trabecular meshwork. This meshwork provides drainage of the fluid through ducts that are distributed around the trabecular annulus, however with age these ducts become blocked with cellular debris.

Methods to improve the drainage have hitherto been attempted with either medication or surgical means. A more recent method, known as laser trabeculoplasty, relies on directing a pulsed, focused laser beam onto the trabecular meshwork with sufficient intensity that pigmented melanin cells suffer damage and initiate biological changes whereby laser-damaged sites are repopulated by cells from a non-filtering region of the trabecular meshwork. These have been found to serve as stem cells producing fresh and functioning cells that have been found to restore the drainage by the trabecular meshwork.

Currently, delivery of a laser beam to the trabecular meshwork is achieved by directing a laser beam obliquely through the cornea of an eye with the aid of an optical element placed in contact with the eye, as shown in FIG. 1. The element includes a mirror to direct the laser beam sideways to the trabecular meshwork. This treatment method is known as selective laser trabeculoplasty or SLT. With this system an ophthalmic practitioner is required to rotate the optical element, known as a gonioscope to treat multiple regions around the trabecular meshwork. When sufficient intensity is achieved, the reaction can be identified by production of micro-bubbles on the surface of the trabecular meshwork.

The shortcomings of this method are several-fold: It can be difficult for a practitioner to accurately direct the beam to a desired spot on the trabecular meshwork (TM); the procedure requires great skill to avoid risks of injury; it may not be feasible to perform the procedure in all patients due to a variety of conditions such a hazy or opacified cornea, intraocular adhesions of ocular tissue to the TM, deep set eyes such that the goniolens cannot be placed onto the eye or difficulty positioning patients, and lastly the procedure can be quite lengthy thereby causing discomfort to the patient.

An improvement to the technique has been proposed in US patent Publication US 2015/0366706 A1 in which a treatment laser beam is directed at the trabecular meshwork through the sclera, as shown in FIG. 2. This proposed method is known as direct selective laser trabeculoplasty or DSLT. DSLT has been proposed as a method to overcome the main disadvantages of SLT, namely the necessity of the placing of a gonioscope on the cornea of the patient's eye to administer the treatment laser to the trabecular meshwork and the skill required to perform the procedure.

However, current DSLT treatment methods suffer from various problems. For example, DSLT only estimates the position of the TM and conjunctival/scleral corneal junction by using photographic methods without direct visualization. Moreover, the healthcare industry is always looking for improvements in treatment protocols for eye disease, such as Glaucoma.

### DESCRIPTION OF THE DRAWINGS

The foregoing aspects and many of the attendant advantages of the claimed subject matter will become more readily appreciated as the same become better understood by reference to the following detailed description, when taken in conjunction with the accompanying drawings, wherein:

FIG. 1 is a schematic diagram showing a treatment method known as selective laser trabeculoplasty (SLT);

FIG. 2 is a schematic diagram showing a treatment method known as direct selective laser trabeculoplasty (DSLT);

FIG. 3 depicts a cross-section of a human eye featuring a trabecular network;

FIG. 4 depicts a front view of a human eye;

FIG. 5 is a perspective view of an example embodiment of a direct view scleral contact lens suitable for use in DLST in accordance with an aspect of the present disclosure;

FIG. 6 is an exploded view of the contact lens of FIG. 5;

FIG. 7 is a cross-sectional view of the contact lens taking along the lines 7-7 shown in FIG. 5;

FIG. 8 is a partial, detailed view of the contact lens of FIG. 7; and

FIG. 9 is a partial, detailed front view of the contact lens of FIG. 5.

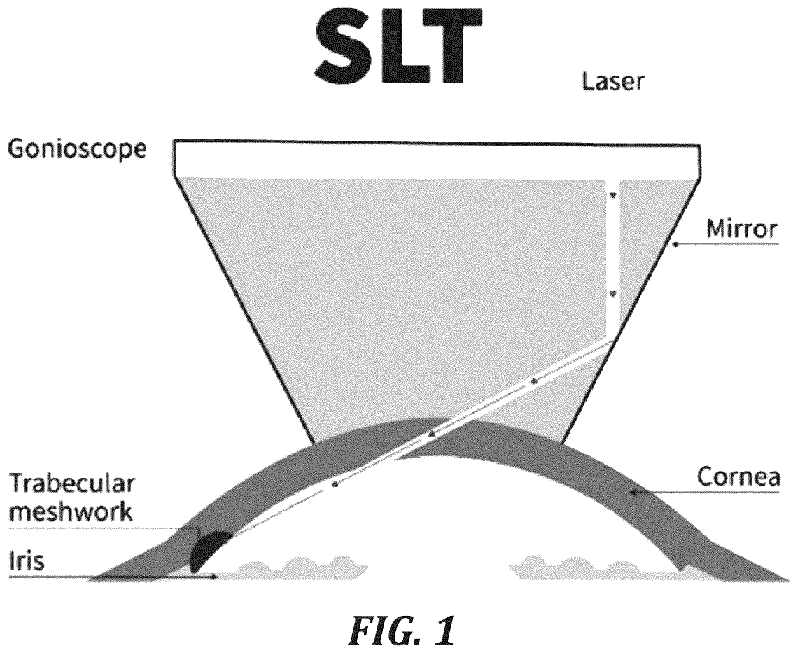

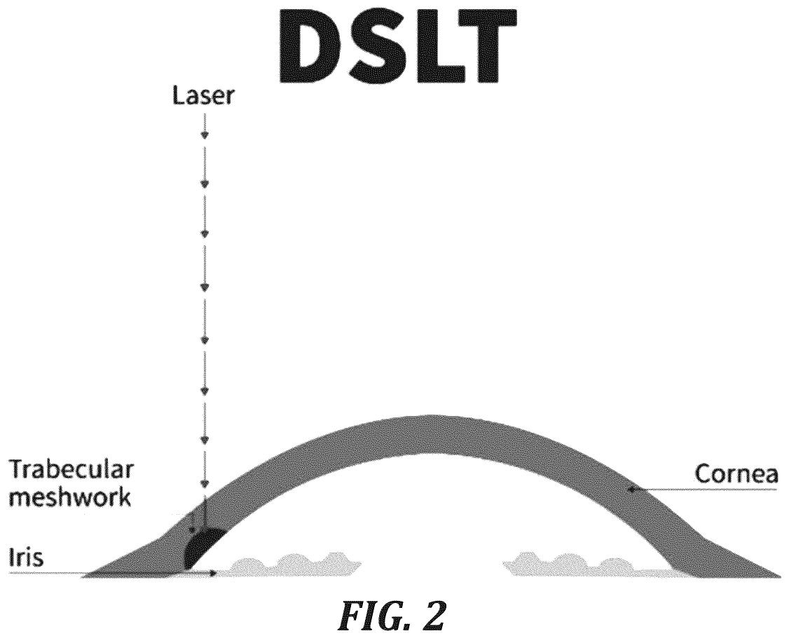

### DETAILED DESCRIPTION

The detailed description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

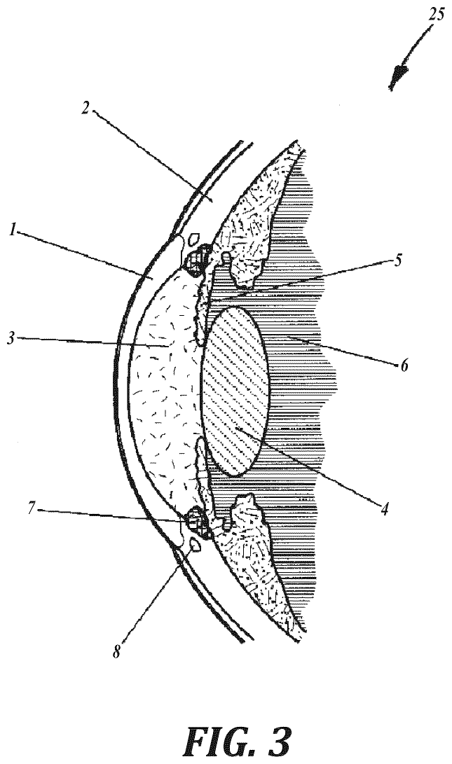

Referring to FIG. 3, a cornea 1 of a left eye 25 of a patient (not shown) connects to a sclera 2. The fluid-filled anterior chamber 3 is contained by the pigment epithelium or iris 5 that surrounds the lens 4. The posterior chamber 6 contains vitreous humor and represents the largest volume of an eye. Nestled between the outer edges of the cornea and the iris is the trabecular meshwork 7, through which drainage is effected into Schlem's canal 8. The trabecular meshwork 7 has a triangular cross-section.

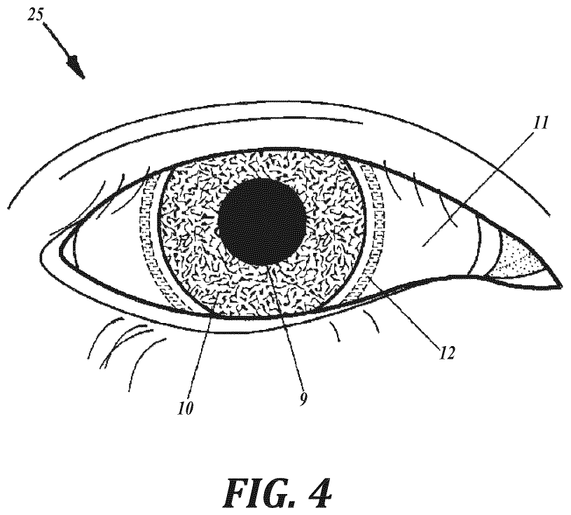

FIG. 4 shows a left eye 25 as it might be presented to a practitioner. It shows a pupil 9 surrounded by an iris 10. The adjacent white sclera 11 conceals a trabecular meshwork 12, shown having exaggerated width within dashed lines. The width of this meshwork 12 is typically in the order of 350 microns with a depth of 50-150 microns.

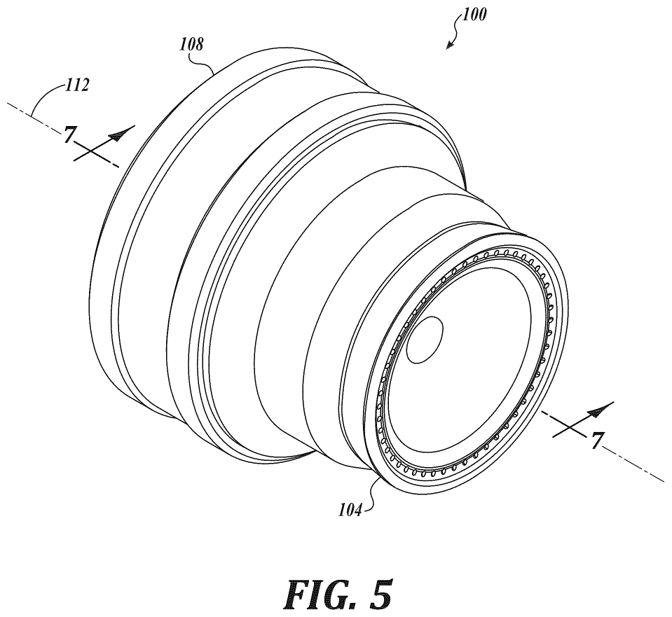

Turning now to FIGS. 5-7, there is shown an example embodiment of a direct view sclera contact lens, generally designated 100, formed in accordance with one or more aspects of the present disclosure. As will be described in more detail below, and against the teachings of the conventional techniques of DSLT, the inventors of the present application believe that a direct view sclera contact lens, such as contact lens 100, is beneficial for DSLT procedures. With use of the contact lens 100, DSLT can be carried out with conventional SLT lasers that are commonly available. Conventional DSLT devices, such as those described in US patent Publication US 2015/0366706 A1, are not commonly available, are expensive, and can only be used for one procedure. On the other hand, lasers typically used for SLT are useful for other therapeutic procedures as well.

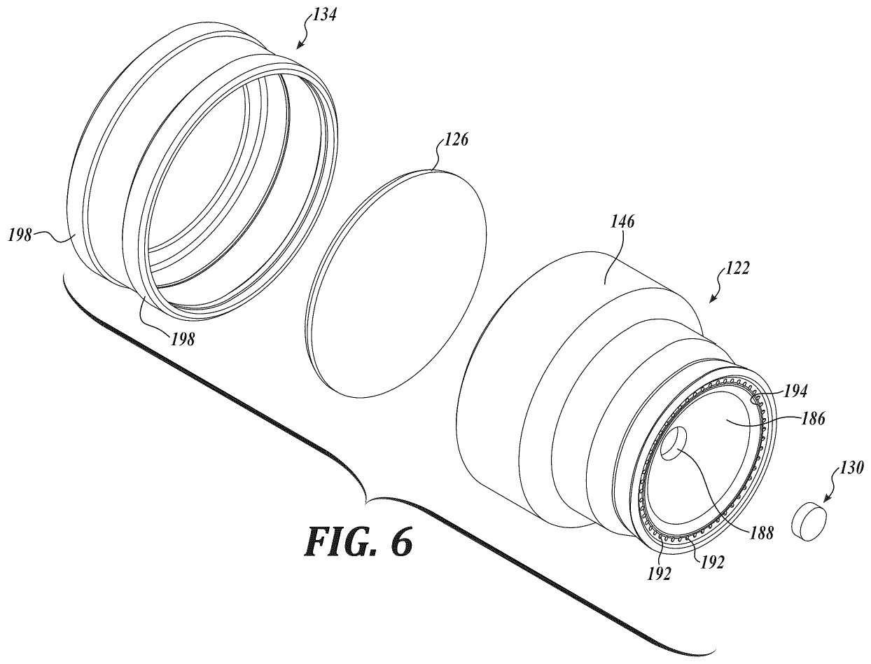

As shown in FIG. 5, the contact lens 100 has a contact end 104 that comes into contact with the eye of the human patient and a non-contact end 108 diametrically opposite the contact end in a direction along the optical axis 112 of the contact lens 100. Referring now to FIG. 6, there is shown an exploded view of the contact lens 100 of FIG. 5. As shown in FIG. 6, the contact lens 100 includes a lens 122, a large transparent disc 126 positioned on the non-contact end side of the lens 122, a small occluding disc 130 positioned the contact end side of the lens 122, and an outer lens holder 134 that surrounds an upper portion of the lens 122 as assembled, as will be described in more detail below.

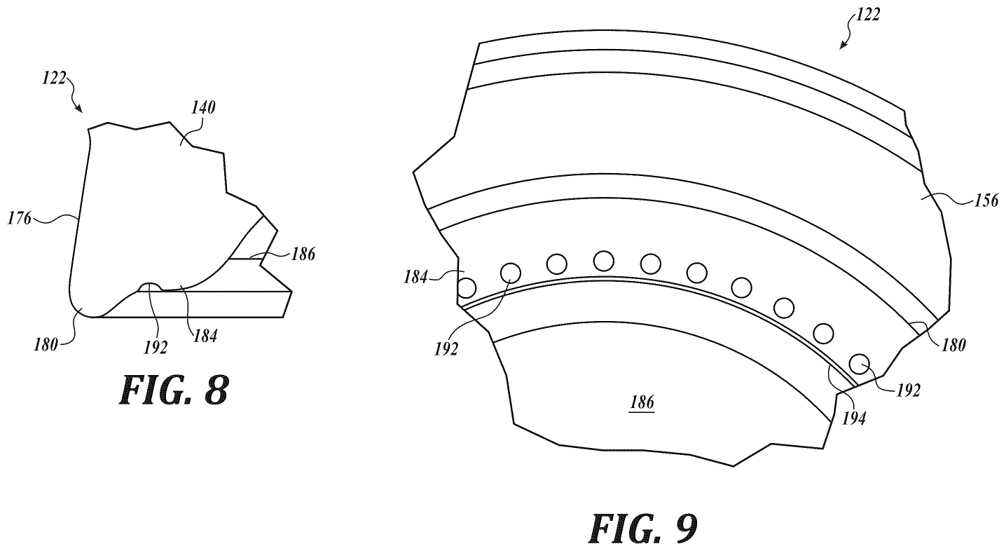

With reference now to FIGS. 5-8, each of the components of the contact lens 100 will be described in more detail. As shown in FIGS. 5-8, the lens 122 has a solid lens body 140 composed of polymethylmethacrylate or other suitable optical material, such as glass, etc., that transmits light in the 350 nm to 1500 nm range. The lens body 140 defines a light entry surface 144 that is planar and is oriented orthogonally to the optical axis 112 of the lens 122. A first cylindrical section 148 of the lens body 140 is formed from the peripheral and superior facing edge of the light entry surface 144. Of course, the intersection of the light entry surface 144 and the first cylindrical section 148 may be formed with a chamfer or fillet, for example. As will be described in more detail below, the outer lens holder 134 fits over a majority of the first cylindrical section 148 of the lens body 140 and is secured thereto with, for example, a suitable adhesive or by means of threads.

Below the outer lens holder 134, the exterior surface 152 of the lens body 140 transitions to a middle section 156 that has a frustoconical shape tapering inferiorly and inwardly toward the optical axis 112. Adjoining the middle section 156 inferiorly is a second cylindrical section 160 that is stepped down (i.e., has a smaller diameter) from the first cylindrical section 148. The exterior surface of the second cylindrical section 160 is generally parallel with the exterior surface of the first cylindrical surface and the optical axis 112. Below the second cylindrical section 160, the exterior surface of the lens body 140 transitions to an end region 168 that has a superior section 172 with a frustoconical shape tapering inferiorly and inwardly toward the optical axis 112 and an inferior section 176 with a frustoconical shape tapering inferiorly and outwardly toward the optical axis 112.

The exterior surface of the inferior section 176 terminates as an annular, outer rim or flange 180. In an embodiment, the outer flange has a diameter of about 17.00 mm. The outer flange 180 is rounded (and somewhat inferiorly facing) and forms the outer border of a light exiting surface 182 of the lens 100. The light exiting surface 182 extends inwardly toward the optical axis 112 and in a superior direction to form a compression annulus 184. The compression annulus 184 is configured for improved compression and blanching of the conjunctival tissue when the lens 100 is pressed against the patient's eye. For example, when the lens 100 is in contact with the patient's eye, the compression annulus provides a more localized annulus of compression located over the trabecular meshwork in the average human eye. At the same time, the outer flange 180 aims to improve eyelid control while not interfering with visualization of the treatment site. It is believed that the compression annulus 184 helps to improve transmission of the light and reduce absorption of laser energy by the limbal vasculature.

A first cavity 186 in the form of a spherical recess is formed inwardly of the light exit surface 182. In that regard, the first cavity 186 extends in a superior direction from the inner rounded edge of the compression annulus 184 and is centered on the optical axis 112 of the lens. In an embodiment, the inner rounded edge has a radius of curvature of about 1.00 mm 1.20 mm, and is on the order of 1.08 mm in a certain embodiment. The radius of curvature of the first cavity 186 is slightly less than the radius of the cornea of the human eye. In an embodiment, the radius of curvature can range from about 6.0 mm to about 9.0 mm. In a certain embodiment, the radius of curvature is on the order of 7.45 mm, and in another certain embodiment, the radius of curvature is on the order of 8.40 mm. In an embodiment, the diameter of the of the first cavity is about 12.00 mm-12.20 mm.

A second cavity 188 having cylindrical sides with an axis coincident with the optical axis 1112 extends in a superior direction from the first cavity 186. In an embodiment, the superior surface of the second cavity 188 may be also concave having a radius corresponding with the first cavity 28. In an embodiment, the second cavity has a diameter of about 3.0 mm-7.0 mm. In a certain embodiment, the diameter of the second cavity is on the order of 3.05 mm. The second or upper cavity 188 is configured to receive therein the occluding disc 130. The occluding disc 130 is formed with an opaque material and in one embodiment can be cast in place with polymethylmethacrylate material. In any case, the occluding disc 130 is retained in place within the second cavity 188. In use, the occluding disc 130 aims to prevent light from exiting the lens 100 and entering the optical portion of the eye.

Referring now to FIGS. 6, 8 and 9, a plurality of guide spots or alignment markers 192 are etched or otherwise formed into the bottom surface (e.g., the light exit surface 192) of the lens body 140. In the embodiment shown, a set of at least 50 alignment makers 192 are evenly spaced to form a ring of alignment markers and are disposed, for example, in the compression annulus 184. In an embodiment, these alignment markers 192 are somewhat spherical in shape, having a diameter and radius of about 0.25 mm-0.40 mm and about 0.15 mm-0.25 mm, respectively. The alignment markers 192 are positioned outwardly about 7.25 mm from the optical axis 112, a location that is exteriorly of the limbus when the lens is on the patient's eye. In a certain embodiment, the alignment markers 192 have a diameter and radius of about 0.34 mm and about 0.19 mm, respectively.

In the embodiment shown, an (optional) annular groove 194 is also etched or otherwise formed inwardly of the ring of alignment markers 192 and adjacent the outer rounded edge of the first cavity 186. In an embodiment, the annular groove 194 has an inside diameter of about 13.72 mm, a width of about 0.07 mm, a depth of about 0.05 mm and is centered on the optical axis 112. These markers 192 and the groove 194 assist the ophthalmologist using the lens 100 in properly aiming and positioning each of the laser applications during the DSLT procedure.

Returning now to FIGS. 5-7, the outer lens holder 134 is formed from a black plastic and surrounds the first cylindrical section 148. The outer lens holder 134 is secured to the lens body 140 with a suitable adhesive or by means of threads (not shown). The outer lens holder 134 includes an inwardly extending annular lip 196 and first and second spaced-apart bands 198. In an embodiment, the first and second bands 198 include knurled outer surfaces to improve the ophthalmologist's grip on the lens 100 during the DSLT procedure.

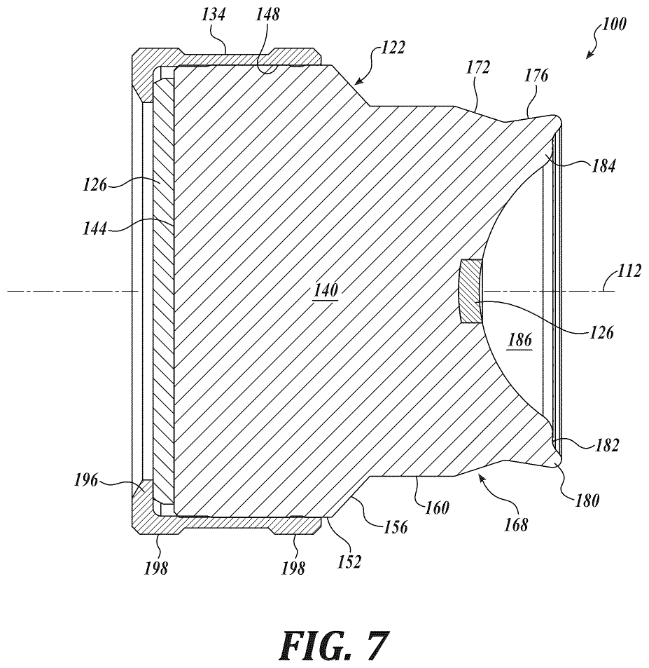

In the embodiment shown and assembled in FIG. 7, the large transparent disc 126 (with an optional anti-reflective coating), is positioned on the light entry surface 144 of the lens 140 and under the inwardly extending annular lip 196. In an embodiment, the large disc 126 can be held in place by the outer lens holder 134. Alternatively or additionally, the large disc 126 can be secured to the lens body 140 via suitable adhesive.

As set forth above, the contact lens 100 is advantageously employed in DSLT procedures. One example of a method for using the contact lens 100 during a DSLT procedure will now be described in more detail.

To begin, a goniogel or like substance is applied to the eye contacting surfaces of the contact lens 100 (e.g., the light exit surface 182, the compression annulus 184, etc.), and with the goniogel applied, the contact lens 100 is placed directedly onto the surface (e.g., cornea, conjunctiva, sclera, etc.) of the eye to be treated. Via the goniogel, the contact lens 100 is optically mated to the surface of the eye to the treated. In an optional step, an topical anesthetic may be applied to the surface of eye to be treated for reducing possible discomfort of the patient during the procedure.

Next, a SLT laser beam is directed through the lens at the junction of the conjunctiva/sclera and cornea, sometimes referred to as the conjunctival/scleral corneal junction. During the procedure, the conjunctival/scleral corneal junction is compressed by the compression annulus 184 of the contact lens 100 and the laser is roughly positioned over the trabecular meshwork below.

As set forth above, any conventional SLT laser can be used. In an embodiment, the SLT laser is a Q-switched frequency-doubled Nd: YAG laser. In one embodiment, the SLT laser is selected from one of the Lumenis Selecta® line of SLT lasers. In an embodiment, the Lumenis Selecta® Trio™ SLT laser may be used. In a certain embodiment usings a Lumenis Selecta® Trio™ SLT laser, the SLT laser has a wavelength of about 532 nm, a pulse duration of about 3 ns (nanoseconds) and a spot size of about 400 microns. This SLT laser also allows for adjustment (in 0.1 mJ increments) of laser's energy between 0.3-2.0 mJ per pulse. The physician can adjust the energy of the laser depending on the needs of the patient, etc. Of course, other SLT lasers can be employed in order the practice embodiments of the present disclosure.

Using the alignment markers 192 of the contact lens 100 as a guide, the laser beam of the SLT laser is then annularly applied to treat up to 100 discrete spots, preferably around the limbus, thereby creating a treatment band. Energy of about 1.8 mJ to about 2.2 mJ is applied by the laser at each spot (i.e., location) of the trabecular meshwork. In other words, laser energy travels through the lens 140, out of the light exiting surface 182, for example the compression annulus 184, and penetrates the tissue overlying the trabecular meshwork. During this time, the contact lens 100 can be adjusted as needed such that the compression annulus 184 continues to compress the limbal vessels.

Once the number of locations (e.g., up to 50, up to 100, etc.) around the trabecular meshwork have been treated with the laser, the contact lens 100 can be removed.

The following non-limiting advantages of using the contact lens 100 of the present disclosure in a DSLT procedure can be realized:
1) The contact lens 100 stabilizes the eye during the procedure;
2) The contact lens 100 compresses the conjunctival vessels at the limbus improving laser transmission through the sclera;
3) The contact lens improves the localization of the laser beam overlying the trabecular meshwork, by direct visualization of the conjunctival/scleral corneal junction; and
4) The contact lens 100 can be used in patients with difficulty positioning, deep orbits which can prevent adequate visualization of the trabecular meshwork, patients with corneal opacification or haze, angle closure with synechiae to the trabecular meshwork, any patient with poor visualization of the trabecular meshwork, patients with tremors, etc.

In the foregoing description, specific details are set forth to provide a thorough understanding of representative embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure.

Although the method and various embodiments thereof have been described as performing sequential steps, the claimed subject matter is not intended to be so limited. As nonlimiting examples, the described steps need not be performed in the described sequence and/or not all steps are required to perform the method. Moreover, embodiments are contemplated in which various steps are performed in parallel, in series, and/or a combination thereof. As such, one of ordinary skill will appreciate that such examples are within the scope of the claimed embodiments.

In the detailed description herein, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. In addition, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments. Thus, it will be appreciated that embodiments of the present disclosure may employ any combination of features described herein. All such combinations or sub-combinations of features are within the scope of the present disclosure.

Throughout this specification, terms of art may be used. These terms are to take on their ordinary meaning in the art from which they come, unless specifically defined herein or the context of their use would clearly suggest otherwise.

The drawings in the FIGURES are not to scale. Similar elements are generally denoted by similar references in the FIGURES. For the purposes of this disclosure, the same or similar elements may bear the same references. Furthermore, the presence of reference numbers or letters in the drawings cannot be considered limiting, even when such numbers or letters are indicated in the claims.

The present application may reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A and B” is equivalent to “A and/or B” or vice versa, namely “A” alone, “B” alone or “A and B.”. Similarly, the phrase “at least one of A, B, and C,” for example, means (A), (B), (C), (A and B), (A and C), (B and C), or (A, B, and C), including all further possible permutations when greater than three elements are listed.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may be independently included in the smaller ranges and are also encompassed within the disclosure, subject to any specifically excluded limit in the stated range. While the stated range includes one or both of the limits, ranges excluding either or both of those included limits are also included in the disclosure.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

1. A contact lens for use in direct selective laser trabeculoplasty, the contact lens comprising: a lens body having an optical axis and an inferior end that defines a light exit surface, the light exit surface extending inwardly toward the optical axis and in a superior direction to form a compression annulus, wherein the compression annulus contacts an eye of the patient during direct selective laser trabeculoplasty, and wherein the lens body includes a ring of evenly spaced alignment markers disposed in the light exit surface.

2. A contact lens for use in direct selective laser trabeculoplasty, the contact lens comprising: a lens body that includes a superior end that defines a light entry surface that is planar and is oriented orthogonally to an optical axis of the lens and an inferior end that defines a light exit surface, the light exit surface extending inwardly toward the optical axis and in a superior direction to form a compression annulus, wherein the lens body also includes a first cavity in the form of a spherical recess that extends in a superior direction from the inner rounded edge of the compression annulus, and wherein the lens body further includes a plurality of alignment markers formed into the light exit surface and are arranged in a ring-like pattern.

3. The contact lens of claim 2, wherein the lens body further includes a second cavity having an axis coincident with the optical axis, the second cavity extending in a superior direction from the first cavity, the second cavity being smaller than the first cavity.

4. The contact lens of claim 3, further comprising an occluded disc disposed in the second cavity.

5. The contact lens of claim 2, wherein the exterior surface of an inferior section of the lens body terminates as an annular, outer rim, wherein the rim is rounded and forms the outer border of a light exit surface of the lens body.

6. The contact lens of claim 5, wherein the inferior section of the lens body includes a superior section with a frustoconical shape tapering inferiorly and inwardly toward the optical axis and an inferior section with a frustoconical shape tapering inferiorly and outwardly toward the optical axis.

7. The contact lens of claim 6, wherein a diameter of the inferior section is less than the diameter of the light entry surface.

8. The contact lens of claim 2, further comprising an outer lens holder that fits over a majority of a superior section of the lens body and is secured thereto.

9. A method of performing direct selective laser trabeculoplasty with a contact lens of claim 1, the method comprising:
placing the contact lens onto the surface of an eye to be treated;
compressing the conjunctival/scleral corneal junction with the compression annulus of the contact lens;
using the alignment markers of the contact lens as a guide to orient a selective laser trabeculoplasty laser so that the laser beam produced by the laser travels through the contact lens at the conjunctival/scleral corneal junction; and
annularly applying the laser beam to treat a plurality of discrete spots around the limbus band, thereby creating a treatment band.
